(19) United States
(12) Patent Application Publication
(10) Pub. No.: US 2004/0215210 A1
(43) Pub. Date: Oct. 28, 2004

(54) Clamp for performing circumcisions on newborns and a method of using the same

(76) Inventor: Barry P. Duel, Long Beach, CA (US)

Correspondence Address: Daniel L. Dawes, MYERS DAWES ANDRAS & SHERMAN LLP, Suite 1150, 19900 MacArthur Blvd., Irvine, CA, 92612, US

(21) Appl. No.: 10/422,123

(22) Filed: Apr. 24, 2003

(51) Int. Cl. A61B 17/32

(52) U.S. Cl. 606/118

(57) ABSTRACT

A circumcision device is a unitary combination comprised of a body, a conical bell over which the foreskin is disposed and into which the glans is inserted, a clamping ring, and a shaft coupled to the body and to one the bell is connected. The clamping ring is split and moves between an open and closed locked position. The bell is drawn up into the locked ring to clamp the foreskin. A handle is coupled to the device to position it. A thumb handle is coupled to the shaft for moving the shaft relative to the body. At one or two knife assemblies are rotatably coupled to the shaft. A plurality of skin clips is coupled to one of either the body or shaft and fixed relative to the bell for temporarily holding the foreskin in place against the bell.

### BACKGROUND OF THE INVENTION

1. Field of the Invention

The invention relates to surgical devices and methods, and in particular to a device and method for performing circumcisions on newborn infants.

2. Description of the Prior Art

In 2000, the most recent year for which complete data are available, there were 4,000,000 births in the U.S. Assuming that half of them were boys, and a 60% newborn circumcision rate (a somewhat conservative estimate), there are about 1,200,000 newborns circumcised in this country annually. All of these are done with some sort of clamp device. In addition, the Muslim world practices routine circumcision as well, which represents about another several million boys born annually.

All newborn circumcisions are done using some sort of clamp device. These serve to clamp the foreskin prior to cutting it, to prevent bleeding. The most popular devices are the Plastibell, Gomco and Mogen clamps. The Gomco clamp is generally felt to be safest, because it includes a bell to protect the glans, or head of the penis, during the clamping and cutting of the foreskin. However, it is cumbersome, and actually requires assembly on the patient each time it is used. The Mogen simply clamps the foreskin above the glans, and the excess skin is then removed. While it is easier to use than the Gomco, it is not quite as safe, and carries the risk of actually cutting or clamping part of the glans during the procedure, since there is no bell. The Plastibell is essentially a plastic ring which is placed inside the foreskin. A suture is then tied around it, the skin is excised, and the ring falls off in about one week.

The construction of the Gomco clamp shown in FIG. 5 and the development of the bloodless technique by Yellen in 1935 and Brodie in 1939 have encouraged the practice of routine circumcision. These factors have made the operation safer, reduced the chances of infection, and practically eliminated hemorrhage as compared to traditional surgical methods. The Gomco clamp is made up of four relatively heavy parts, which are difficult to manipulate and position during the operation, by a skilled surgeon or Mohel (a nonphysician trained in newborn circumcision consistent with Jewish tradition). The parts are a plate, a stud (bell), an arm (yoke), and a nut (to tighten the clamp). The device is thus expensive to manufacture and requires careful sterilization practices between multiple usages. The bell is introduced into the preputial cavity (over the glans and under the foreskin) and the prepuce is drawn over it. The plate is then placed over the bell so that the prepuce is sandwiched between them. The arm is fitted into its proper place, and when the nut is screwed on tightly, it exerts a crushing force on the prepuce at the junction of the bell and plate. The clamp is left on for 5 minutes to achieve hemostasis, and the prepuce is excised.

Another prior art device is called the Mogen clamp shown in FIG. 6. Bronstein, a Brooklyn Mohel, who invented the Mogen clamp, also invented the Nutech clamp. The Mogen Clamp is a variation of the Kantor clamp. Instead of crushing the tissue by the attached hemostat, the Mogen shield acts as the clamp. It has the shortcomings of the Kantor clamp, plus one uniquely its own: with the glans below completely out of sight, there is a chance that the tip of the glans might be caught in the clamp. However, this is the preferred device of the more modern Jewish Mohel, and is newly reported to cause less discomfort during infant circumcision, than does the Gomco clamp.

The Mogen device is used by stretching the preputial opening and breaking the preputial adhesions so that the foreskin is completely retractile using a blunt-edged probe. The prepuce is lifted in an upward and outward direction with a hemostat. This action should cause the glans to retract towards the scrotum, preventing accidental amputation of the glans. The open jaws of the Mogen clamp are placed around the prepuce (grooved side facing the glans) and lifted upwards. The clamp is closed. It should be left closed for one to one and one-half minutes. If the infant is more than 6 months old it should remain closed for no less than five minutes. While the clamp is closed, the prepuce is excised distal to the clamp.

Yet another prior art device is the Plastibell made by Hollister shown in FIG. 7. The Plastibell is a plastic bell with a groove close to the edge. It is a disposable plastic device. The bell is inserted into the preputial cavity (over the glans, and under the foreskin) and the foreskin (prepuce) is tied around it with a tight string. Blood flow to the prepuce is ceased, and the prepuce forward of the string is cut off. After several days, the prepuce caught under the string necrotizes and falls off, providing a bloodless circumcision, with no open wound to become irritated or infected.

While all circumcision devices are meant to simplify the operation and to increase the safety of the operation, use of the devices must nonetheless be done with knowledge of the limitations of the devices, which includes their appropriate use, particularly when being used by a single operator. For example, because of the danger of the plastic ring being pulled back behind the glans, only the foreskin that naturally covers the glans can be safely removed using the Plastibell. The Plastibell is primarily used on infants in the United States, although youth, teen and adult models are sometimes used in Europe. The Plastibell is used in nearly 60% of all routine infant circumcisions in the US with 39% being done by the Gomco clamp in the United States. Both the Gomco Clamp and the Plastibell, if used in accordance with the maker's recommendations, crush the foreskin. The biggest difference is that the Plastibell works entirely by this method whereas the Gomco crushes only until the unwanted skin is cut away to perform the actual circumcision. In theory either should produce equally cosmetic results.

The procedure is completed and the Plastibell is in place and will remain on the boy's penis for approximately 7 days before falling off (usually in the bath). Because the Plastibell remains on the penis for 5-10 days whilst it does its job, and is pulled tight against the glans by the foreskin trying to retract back to its unstretched position, Hollister recommends that the foreskin should not be pulled too far forward before being trapped by the thread around the Plastibell's groove. This results in a necessarily looser circumcision than can be achieved with the Gomco clamp. Furthermore, the position of the scar line behind the glans is determined only by the distance from the back of the glans to the groove in the bell. For a given glans circumference (and hence Plastibell size) the longer the boy's glans the further back the resulting scar line will be and the looser the circumcision will be.

If the Plastibell is fixed too tightly then the front of it can dig into the glans and cause problems, including obstruction of the urethra, because it is there for a week or so. The step of clamping the foreskin to the handle of the bell seems to me to be one which doctors are compelled to take if working alone—otherwise there is no way of ensuring that the foreskin stays in place over the bell whilst tying the knots to perform the circumcision proper. If there is an assistant doctor (or nurse) present then they can hold the end of the foreskin in forceps whilst the surgeon ties the knot. This step of keeping the foreskin pulled forward is essential if the Plastibell is to remove an adequate amount of foreskin.

However, the instructions for the Plastibell explicitly state that the skin should not be pulled too tight before being tied off. This is because if pulled very tight there will be considerable tension backwards after the bell has been tied in place. The end of the bell will be pulled into the glans and may easily compress the urethra to the extent of making urination very painful, if not impossible. A wise surgeon passes a probe down the urethra after fixing the bell so as to prove that it is not obstructed. Only then does he finally cut the thread to length.

As mentioned above, there are many different types of circumcision clamps. The Gomco is safest, since it incorporates a bell to protect the head of the penis (glans). However, it is very cumbersome, requiring assembly each time it is used. The Mogen is rapid and effective, but does not incorporate any method to protect the glans, and injuries are possible with this device, some quite severe. The Plastibell is safe and effective, but requires that a large stitch and plastic ring remain on the child's penis for 7-10 days. Understandably, many parents find this distressing.

What is needed is some is some type of clamp that has the advantages of the prior art devices describe above, but which is pre-assembled, making it much easier to use than, and which is safer than a Mogen clamp. In addition, it should include an integrated cutting blade, eliminating the additional need for a scalpel.

### BRIEF SUMMARY OF THE INVENTION

The invention is an apparatus for performing circumcision of a foreskin and adapted for unassisted use by a user. The apparatus comprises a body, a conical bell over which the foreskin is disposed and into which the glans is inserted, a clamping ring, and a shaft coupled to the body and to one of either the bell or clamping ring. Regardless of whether the bell is fixed to the body or the shaft, the variable disposition between the bell and the clamping ring provides for varying clearance between the clamping ring and bell to allow for clamping of the foreskin against the bell to achieve hemostasis. In other words, the bell can be held fixed and the clamping ring brought by the shaft downward into contact with the outer conical surface of the bell to clamp the foreskin between them; or the clamping ring can be held fixed and the bell drawn upwardly by the shaft into contact with the clamping ring to clamp the foreskin between the ring and bell. In the preferred embodiment the clamping ring is stationary and bell is moved by means of the shaft. Where the clamping ring is stationary and the bell moves, the clamping ring is split and moves between an open and closed locked position. However, where the bell is stationary and the clamping ring moves, it is possible that the clamping ring will be a fixed or closed circle.

The body, bell, clamping ring and shaft comprise a unitary combination during use, i.e. it normally does not need to be assembled or disassembled to be used.

Preferably, the bell is rotatably coupled to the shaft. A handle is coupled to the body to facilitate positioning of the body with a single hand. A thumb handle is coupled to the shaft for moving the shaft relative to the body. At least one and preferably two knife assemblies are rotatably coupled to the shaft.

At least one and preferably a plurality of skin clip is coupled to one of either the body or shaft and fixed relative to the bell for temporarily holding the foreskin in place against the bell.

In the illustrated embodiment the clamping ring is comprised to two half-circle clamps, which includes a catch for locking the two half-circle clamps together.

The body, bell, clamping ring and shaft combine to form a disposable or nondisposable combination.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a perspective view of the illustrated embodiment of the invention.

FIG. 2 is side elevational view of the illustrated embodiment of the invention.

FIG. 3 is front elevational view of the illustrated embodiment of the invention.

FIG. 4 is a perspective view of another embodiment of the invention.

FIG. 5 is a depiction of the prior art Gomco clamp.

FIG. 6 is a depiction of the prior art Mogen clamp.

FIG. 7 is a depiction of the prior art Plastibell clamp.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

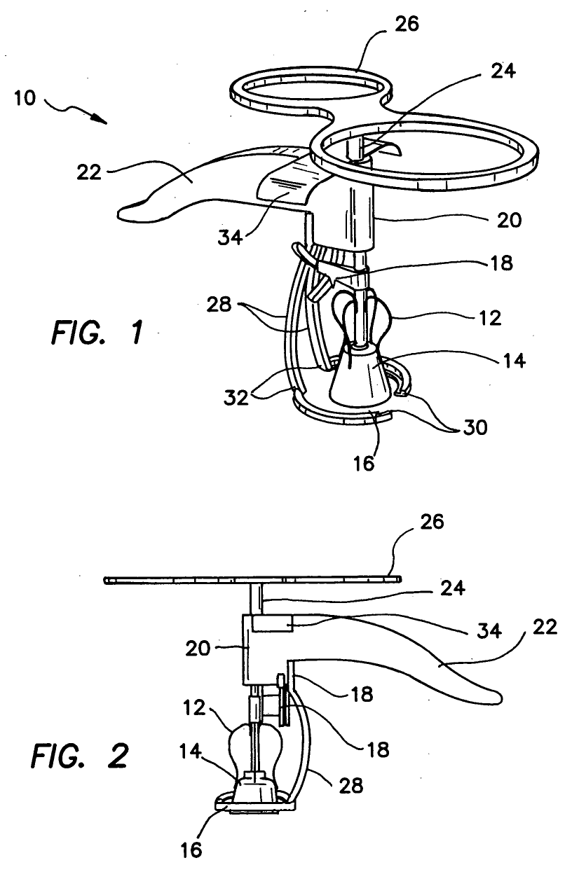

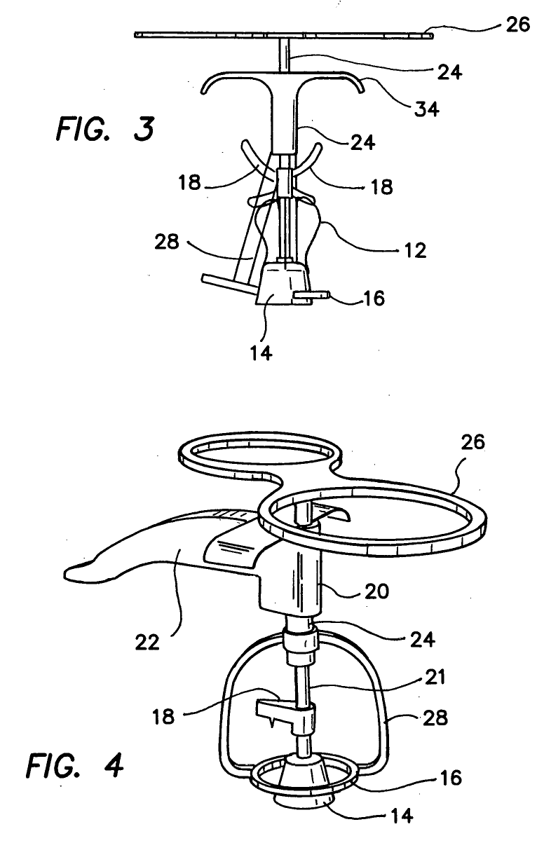

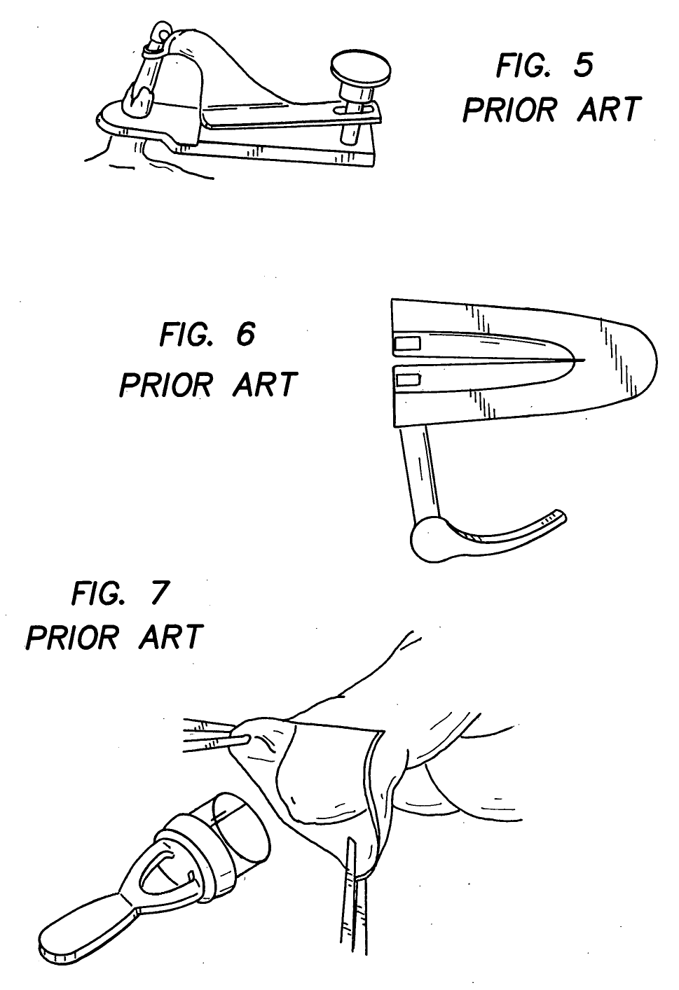

The circumcision clamp of the invention, known as the Duel Clamp, and generally denoted by reference numeral 10, is shown in perspective view in FIG. 1. Clamp 10 is comprised of a body 20 from which a fixed handle 22 extends. Handle 22 may have any form desired to facilitate the positioning or holding of clamp 10 during use. In the illustrated embodiment they include a transverse horizontal fixed wing 34 best seen in the front view of FIG. 3 to assist in firm gripping or engagement of handle 22 with the fingers of one hand. A threaded shaft 24 is engaged in a threaded bore defined in body 20 and can be rotated in body 20 to vertically advance and retract shaft 24 relative to body 20. While shaft 24 is preferably threaded for this purpose, any other mechanism or arrangement may be equivalently substituted such as a rack and pinion combination, slide and wedge combination or any other means for relative movement now known or later devised.

Shaft 24 is coupled to thumb handles 26 which form a symmetrical figure eight and allow the surgeon to rotate shaft 24 in either sense with two fingers of one hand while holding body 20 stationary by means of handle 22 with the other hand. Again the particular form of thumb handles 26 as shown in FIG. 1 is not material to the invention and other means for rotation of shaft 24 can be substituted.

A pair of clamp arms 28 are coupled to body 20 at their upper end as shown in FIG. 1 and carry a corresponding pair of half-circle clamps 16 at their opposing lower end. One or both of clamp arms 28 are rotatable about their upper coupling to body 20. The lower ends 32 of clamp arms 28 are each fixed to one of the half-circle clamps 16. The opposing end of the half-circle clamps 16 are provided with a catch 30, so that when half-circle clamps 16 are moved together catch 30 engages on each opposing end of the half-circle clamps 16 and locks half-circle clamps 16 into the configuration of a rigid closed circle or ring. Clamp arms 28 may be spring-biased toward their open position by conventional means (not shown), may be freely rotatable relative to body 20 but friction stabilized, or may be coupled to a sliding collar (not shown) riding on shaft 24 by a cam action to close or rotate clamp arms 28 together.

A hollow bell 14 is coupled to and freely rotatable on the lower end of shaft 24 as seen in FIG. 1. Half-circle clamps 16 are positioned relative to the throw of shaft 24 such that bell 14 is disposed inside of the rigid closed circle or ring of half-circle clamps 16 at variously adjustable vertical positions as best seen in the side elevational view of FIG. 2 or the front elevational view of FIG. 3. Bell 14 has a truncated conical shape so that the amount of clearance between bell 14 and the rigid closed circle or ring of half-circle clamps 16 can be adjusted by adjusting the throw of shaft 24 and hence the vertical position of bell 14 in the rigid closed circle or ring of half-circle clamps 16.

A pair of knife assemblies 18 each having its own knife edge as best shown in FIGS. 2 and 3 may be rotatably and threadably coupled to shaft 24 and brought to bear against the foreskin stretched over bell 14 and held in place by means of the rigid closed circle or ring of half-circle clamps 16 as described below. A pair of knife assemblies 18 would be employed to each cut at least an 180° incision in the case where a 360° were not possible due to the interference of clamp arms 28. It is of course to be understood that a single knife assembly could be used were 360° were possible.

Alternatively, knife assemblies 18 could be omitted and a handheld scalpel used as is the traditional practice, but the use of knife assemblies 18 is preferred since this allows for ease of manipulation and use of clamp 10 during the operation by an unassisted surgeon and insures a fixed and safe path for the incision cut by the knife edge in all cases.

In another embodiment of the invention, clamp 10 is provided with a plurality of spring clips 12 which extend over bell 14 and provide a means for temporarily holding the foreskin in place when it is stretched over bell 14 and as knife assemblies 18 are brought to bear. The plurality of spring clips 12 are attached to shaft 24 at one end, such as by being formed into an eyelet or ring end through which shaft 24 extends, and extend outwardly over knife assemblies 18 in an arc to bring their distal ends resiliently against bell 14. The arc of spring clips 12 is sufficient to allow clearance for knife assemblies 18 which are rotated and lowered around shaft 24 beneath spring clips 12.

The most difficult part of assembling a Gomco clamp is pulling the bell and foreskin through the hole in the baseplate, then assembling the entire device. The clamp 10 of the invention eliminates these problems. First, the clamp 10 is pre-assembled, making its use much easier. In addition, clamp 10 has clips 12 to keep the foreskin in place on the bell 14. Second, the baseplate of the Gomco is replaced with the half-circle clamps 16, which then clamp around the foreskin and tighten down to the bell 14. This makes for a much quicker procedure. Third, clamp 10 can incorporate cutting blades 18, eliminating the additional need for a scalpel.

Clamp 10 may be disposable plastic unit or non-disposable sterilizable metallic unit. In either case, clamp 10 is integral in the sense that no element or piece has to be assembled in order for clamp 10 to be used or the operation using clamp 10 performed. Further, the use of clamp 10 never requires the use of more than two hands. Clamp 10 is held in place with one hand during the operation and the clamping action of half-circle clamps 16 and bell 14, and the cutting of blades 18 performed using the other hand. In the case of a non-disposable version, the half-circle clamps 16, cutting blades 18 and bell 14 could be removable and manufactured in different sizes, which would reduce costs versus an entirely disposable version.

The structure of clamp 10 having been described above, consider now the use of clamp 10. For use, a slit is made in the dorsal surface of the foreskin, and bell 14 is placed over the glans within the foreskin. The spring clips 12 are used to keep the foreskin in position during the procedure. The half-circle clamps 16 are then brought together locking in place with catch or ratchets 30, forming a complete circle around the foreskin, analogous to the baseplate of a Gomco clamp. Hemostasis is then achieved in this locked configuration. The thumb handles 26 are then screwed down. In the preferred embodiment clamp arms 28 are attached to a sliding collar (not shown) on the threaded shaft 24, so movement of the collar down shaft 24 forces the half-circle clamps 16 and bell 14 together, pinching the foreskin circumferentially. The cutting blades 18, if attached, are then lowered onto the foreskin stretched over bell 14 , and are used to cut the skin by rotating cutting blades 18 in opposite directions. The half-circle clamps 16 are then released, and the entire clamp 10 is removed. Thus clamp 10 is used as a unitary device during the operation and is disengaged from the patient as a unitary device. In the case of the Gomco device, the unit must be held by an assistant at some points during the operation or left attached unsupported hanging from the patient. Given the size and weight of the Gomco device and the small size of newborns, this option is often impractical or undesirable.

The preferred embodiment has been shown in and described in FIGS. 1-3 has including a movable bell 14 and a fixed, but openable and closeable clamping ring 16, which cinches up against bell 14. The relative roles of bell 14 and clamping ring 16 could also be reversed without departing from the spirit and scope of the invention. For example, in the embodiment of FIG. 4 clamping ring 16 is a fixed or unbroken ring which is cinched up against bell 14, but bell 14 is fixed relative to body 24 by being connected thereto by a rod 21, which is telescopically disposed into hollow shaft 14 and fixed to body 20. Clamping ring 16 is then connected, by arms 28 with which it may be integral to threaded shaft 24, which rotates in body 24 to lower or raise clamping ring 16 relative to bell 14. Arms 28 may be fixed or swiveled to shaft 24. Knife assembly 18 slides on rod 21 and is rotatable about it.

Alternatively, shaft 24 and rod 21 may both integral with each other and fixed to body 20 with arms 28 threadably connected to shaft 24 and rotatable about it. In this case, thumb handle 26 may be omitted and threaded shaft 24 fixed relative to body 20.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. An apparatus for performing circumcision of a foreskin and adapted for unassisted use by a user comprising:
a body;
a conical bell over which the foreskin is disposed;
a clamping ring; and
a shaft coupled to the body and to one of either the bell or clamping ring for providing variable disposition between the bell and the clamping ring, the other one of the bell or clamping ring being coupled to the body, the clamping ring and bell being variably disposable relative to each other to provide for varying clearance between the clamping ring and bell to allow for clamping of the foreskin against the bell to achieve hemostasis, and where the body, bell, clamping ring and shaft comprise a unitary combination during use.

2. The apparatus of claim 1 where the bell is coupled to the shaft, and the clamping ring is coupled to the body.

3. The apparatus of claim 2 where the bell is rotatably coupled to the shaft.

4. The apparatus of claim 1 where the bell is coupled to the body, and the clamping ring is coupled to the shaft.

5. The apparatus of claim 1 further comprising a handle coupled to the body to facilitate positioning of the body with a single hand.

6. The apparatus of claim 1 further comprising a handle coupled to the shaft for moving the shaft relative to the body.

7. The apparatus of claim 6 where the handle comprising a thumb handle.

8. The apparatus of claim 1 further comprising at least one knife assembly rotatably coupled to the shaft.

9. The apparatus of claim 1 further comprising two knife assemblies rotatably coupled to the shaft.

10. The apparatus of claim 1 further comprising at least one skin clip coupled to one of either the body or shaft and fixed relative to the bell for temporarily holding the foreskin in place against the bell.

11. The apparatus of claim 2 where the clamping ring is comprised to two half-circle clamps.

12. The apparatus of claim 11 where the clamping ring is comprised two movable clamp arms, each coupled to one of the two half-circle clamps.

13. The apparatus of claim 11 where the two half-circle clamps includes a catch for locking the two half-circle clamps together.

14. The apparatus of claim 1 where the body, bell, clamping ring and shaft combine to form a disposable combination.

15. The apparatus of claim 1 where the body, bell, clamping ring and shaft combine to form a nondisposable combination.

16. An apparatus for performing circumcision of a foreskin and adapted for unassisted use by a user comprising:
a body;
a handle coupled to the body to facilitate positioning of the body with a single hand;
a pair of clamp arms rotatably coupled to the body;
a pair of half-circle clamps, each one of the pair of half-circle clamps coupled to one of the pair of clamp arms;
a shaft coupled to the body;
a thumb handle coupled to the shaft for moving the shaft relative to the body;
at least one knife assembly rotatably coupled to the shaft; and
a conical bell coupled to the shaft over which the foreskin is disposed, where the shaft provides for variable disposition between the bell and the pair of half-circle clamps for varying clearance between the clamping ring and bell to allow for clamping of the foreskin against the bell to achieve hemostasis, and where the body, bell, clamping ring and shaft comprise a unitary combination during use.

17. The apparatus of claim 15 where relative movement of the shaft with respect to the body opens or closes the pair of arms to open or close the pair of half-circle clamps.

18. The apparatus of claim 15 where the bell is rotatably coupled to the shaft.

19. The apparatus of claim 15 further comprising at least one skin clip coupled to one of either the body or shaft and fixed relative to the bell for temporarily holding the foreskin in place against the bell.

20. The apparatus of claim 15 where the two half-circle clamps includes a catch for locking the two half-circle clamps together.

21. The apparatus of claim 15 where the body, bell, clamping ring and shaft combine to form a disposable combination.

22. The apparatus of claim 15 where the body, bell, clamping ring and shaft combine to form a nondisposable combination.
